(19) United States
(12) Patent Application Publication
(10) Pub. No.: US 2007/0032781 A1
(43) Pub. Date: Feb. 8, 2007

(54) Reduction of hair growth

(76) Inventors: James P. Henry, Mendon, MA (US); Hsing-Mei Wu, Newton, MA (US); Douglas Shander, Acton, MA (US)

Correspondence Address: FISH & RICHARDSON P.C., P.O. BOX 1022, MINNEAPOLIS, MN, 55440-1022, US

(21) Appl. No.: 11/313,893

(22) Filed: Dec. 21, 2005

Related U.S. Application Data
(60) Provisional application No. 60/639,082, filed on Dec. 22, 2004.

(51) Int. Cl. A61B 18/18

(52) U.S. Cl. 606/9

(57) ABSTRACT

The growth of hair can be reduced by heating the skin sufficiently to retard hair growth but without altering the hair growth cycle.

### CLAIM OF PRIORITY

This application claims priority under 35 U.S.C. §119(e) to U.S. Patent Application Ser. No. 60/639,082 filed on Dec. 22, 2004, the entire contents of which are hereby incorporated by reference.

### BACKGROUND

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth and the hair coarseness. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, ornithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

Various physical methods for temporary or permanent reduction in hair growth by follicle heating depend on a photo-thermal mechanism in which tissue melanin absorbs light to generate large temperature increases resulting in tissue damage associated with protein denaturation. The devices (for example, lasers) used in the methods can raise safety concerns and often require the presence of medical staff during use. In addition, the devices often are more effective with dark hair than with light hair, since dark hair generally includes more melanin.

The hair bulb, isthmus, and infundibulum are the three units that make up the vertical sections of the hair follicle. The hair bulb begins at the base of the hair bulb and continues to the insertion of the arrector pili muscle. This portion of the hair generally is 3-7 mm below the surface of the skin and exists in the dermis. This portion of the hair is made up of the matrix cells, melanocytes, and the dermal papilla. The matrix cells are germinative fibroblasts cells that form the outer root sheath, the three layers of the inner root sheet and the hair shaft and many of the inhibitors of enzyme used to modify hair growth may act on the germinative cell population to decrease the rate of hair growth. The melanin (color) producing cells are also found in the matrix area. The dermal papilla provides the factors responsible for controlling the growth of the hair follicle. The isthmus is found from the insertion of the arrecctor pili muscles to the entrance of the sebaceous duct. The isthmus contains the bulge region of the hair follicle that contains the pleuripotent stem cells needed to regenerate the hair follicle as it enters the anagen phase from the telogen phase. These phases are discussed further below. The infundibulum is the remainder of the hair follicle.

The hair has three growth phases. The anagen phase is characterized by rapid growth of the matrix cells. These cells form the hair shaft as they move up the follicle and differentiate. This is the longest phase of hair growth and can last from months to years. After the anagen phase the hair enters into a short-lived regressive stage called catagen. During catagen the lower portion of the follicle including the melanocytes undergo apoptosis and degenerate. The lower portion of the follicles shrinks to a thin epithelial cord, which retracts upward. During catagen the length of the follicle will be reduced significantly (for example, by about ⅔ of its original length). The dermal papilla will end up at about the level of the arrector pili muscle. Catagen generally lasts about three weeks and is characterized by cessation of mitosis and reabsorption of the lower part of the follicle. The nourishment provided by the papillary blood supply is markedly diminished in this stage and biological approaches to control hair growth include agents affecting the blood supply crucial to maintaining anagen and viable rates of hair growth. The final phase of the hair cycle is the telogen phase or resting phase. This phase can last from weeks to a few months. The hair will re-enter anagen with a large increase in epithelial cell division which will reform the matrix region. The duration of the anagen and telogen phases as well as the numbers of hairs in each phase differ throughout the body. The long hair produced on the scalp is produced by a long anagen phase, whereas hair on the legs is shorter because they spend less time in anagen and more time in telogen.

### SUMMARY

In one aspect, the invention provides a method (typically a cosmetic method) of reducing unwanted mammalian (preferably human) hair growth. The method includes selecting an area of skin on the mammal from which reduced hair growth is desired and heating the area of skin to retard the growth of the hair without altering the growth cycle of the hair. The method does not damage the hair follicle (for example, does not cause significant denaturation of proteins) or the surrounding tissue and does not cause catagen formation.

In another aspect, the invention provides another method (typically a cosmetic method) of reducing unwanted mammalian (preferably human) hair growth. The method includes selecting an area of skin on the mammal from which reduced hair growth is desired, and heating the area of skin to reach a temperature of between 40° C. and 55° C. to cause a reduction in hair growth.

In another aspect, the invention provides another method (typically a cosmetic method) of reducing unwanted mammalian hair growth. The method includes selecting an area of skin on the mammal from which reduced hair growth is desired, and heating the area of skin to a temperature of between 40° C. and 50° C. when measured at about 5 mm below the skin surface, to cause a reduction in hair growth.

The unwanted hair growth may be undesirable from a cosmetic standpoint or may result, for example, from a disease or abnormal condition. The area of skin generally is heated, for at least two, five, or ten seconds. More commonly, the area of skin is heated for at least thirty seconds or a minute, and up to ten minutes or thirty minutes.

The methods can be performed on a daily basis as part of the normal grooming regimen. In a preferred application, the treatment is given at least twice or at least five times in seven days, for example, once a day for at least a week. The frequency and duration of treatment may depend on the skin site from where the hair growth reduction is desired, the type of hair (coarse or fine) and the method of treatment. The methods can be used in conjunction with other hair removal procedures, such as laser hair removal, to prolong the depilatory effect of the procedure.

The methods generally are safe and do not damage, for example, skin or eyes of the mammal. The method typically can be performed without the supervision of medical staff. For example, a heater can be incorporated into a razor and the skin can routinely be heated during shaving. Moreover, depending on the mechanism used for heating, a large area of skin can be heated in one procedure. In addition, depending on the mechanism used for heating, the method can work equally well with dark and light hair, and with dark and light skin. The various ways in which the area of skin can be heated are embodiments of the invention and are discussed in the Detailed Description section.

In some embodiments, the methods further includes applying a dermatologically acceptable composition including a compound that reduces the growth of hair to the area of skin. The compound may be, for example, α-difluoromethylornithine, triptolide, an inhibitor of angiogenesis, or an inhibitor of metalloproteinase.

In another aspect, the invention provides another method (typically a cosmetic method) of reducing unwanted mammalian (preferably human) hair growth. The method includes selecting an area of skin on the mammal from which reduced hair growth is desired, and heating the area of skin to a temperature of between 40° C. and 55° C. to cause a reduction in hair growth.

In another aspect, the invention provides another method (typically a cosmetic method) of reducing unwanted mammalian (preferably human) hair growth. The method includes (a) selecting an area of skin on the mammal from which reduced hair growth is desired; (b) applying to the area of skin a dermatologically acceptable composition including a compound that reduces hair growth; and (c) within 24 hours of step (b), treating the area of skin with a laser, flashlamp, or IPL device operating at sufficiently low energy (for example, less than 10 J/cm², preferably less than 5 J/cm²) that in the absence of applying said composition hair growth would not be noticeably reduced.

In another aspect, the invention provides another method (typically a cosmetic method) of reducing unwanted mammalian (preferably human) hair growth. The method includes (a) selecting an area of skin on the mammal from which reduced hair growth is desired; (b) applying to the area of skin a dermatologically acceptable composition including magnetic oxide nanoparticles; and (c) within a week (for example, within 24 hours or one hour) treating the area of skin with alternating magnetic fields to generate heat. The heat may raise the temperature of the skin and/or hair follicles to, for example, 40° C.-55° C. to gain the benefits described above, or alternatively may raise the temperature of skin and/or hair follicles to a higher temperature that can destroy the hair.

In another aspect, the invention provides another method (typically a cosmetic method) of reducing unwanted mammalian (preferably human) hair growth. The method includes (a) selecting an area of skin on the mammal from which reduced hair growth is desired; (b) applying to the area of skin a dermatologically acceptable composition including nanoshells that absorb infrared light; and (c) within a week (for example, 24 hours or one hour) treating the area of skin with infrared light (preferably near infrared light) to generate heat. The heat may raise the temperature of the skin and/or hair follicles to, for example, 40° C.-55° C. to gain the benefits described above, or alternatively may raise the temperature of skin and/or hair follicles to a higher temperature that can destroy the hair.

Reduced hair growth can be demonstrated quantitatively by reduced hair length, hair diameter, hair pigmentation, and/or hair density in the treated area. Reduced hair growth can be demonstrated cosmetically by less visible hair, shorter hair stubble, finer/thinner hair, softer hair, and/or a longer-lasting shave in the treated area.

Other features, objects, and advantages of the invention will be apparent from the description and drawings, and from the claims.

### DETAILED DESCRIPTION

Various mechanisms can be used to heat the skin to a temperature of between 40° C. and 55° C.

One mechanism is to apply a heating device to the skin. The heating device may be, for example, battery or electric powered. Examples of such devices are described in U.S. Pat. 3,569,666; U.S. Pat. No. 4,021,640; and U.S. Pat. No. 4,950,868. The description of the devices in these patents are incorporated by reference. The device may also simply be an object that itself is heated, for example, in an oven before application to the skin. In some embodiments, where the heating device is battery or electric powered, the device does not also include a cooling unit.

The device may be used, for example, during shaving, and may be incorporated into a dry or wet shaver. The dry shaver itself may be battery or electric powered. For example, the heating device could be part of a shaving/epilating device that uses a battery to heat a resistor that connects to a heating element that contacts the skin.

A laser, flashlamp, or an IPL device may also be used to heat the skin. The laser may be a diode laser, or a solid state laser, for example, a quantum cascade lasers; gas lasers, for example, an ion laser (including metal vapor lasers); and dye lasers. The laser may be operated in the continuous-wave, pulsed, or ultra-fast mode. The laser may be pumped, for example, by electric current, lamp, or another laser. The light may be delivered to the skin by fiber optics or light guide. The laser may include optical filters to reduce the power level. An example of a laser that can be used is a low energy Helium-Neon laser (632.8 nm). Examples of light or photothermal sources that can be used to heat the skin include a diode laser in the wavelength range of 700-1300 nm (e.g., 810±25 nm), a Ruby laser at 654 nm, an Alexandrite laser at 755 nm, a Nd:YAG laser at 1064 nm, a Nd:YAG laser in the range of 600-850 nm, a Pulsed Light, Intense Pulsed Light, or a Flash Lamp in the wavelength range of 400-1200 nm, a Fluorescent Pulsed Light at 550, 580, or 615-1200 nm, a Light Emitting Diode (LED) in the wavelength range of 400-700 nm, and an optical (580-980 nm) or Diode (800+25 nm) energy combined with Radio-Frequency electrical energy. The energy output (J/cm²) of the devices listed above can be, for example, from 0.5-50 J/cm², from 2-20 J/cm², or from 1-10 J/cm².

Other laser and light source parameters that effect the skin and hair follicle heating include, pulse duration, spot size, and repetition rate. The ranges for these parameters depend on the light or the laser source being used. The pulse duration can range, for example, from 0.1 ms to up to 500 ms or it could be a continuous wave (CW), as described in U.S. Pat. No. 6,273,884.

The skin and/or hair follicles also may be heated by applying magnetic oxide nanoparticles to the skin followed by exposing the skin to alternating magnetic fields to generate the heat (Acad Radiol 2002, 9:198-202). Examples of oxide nanoparticles include iron oxides of nontoxic magnetite (Fe₃O₄), ferromagnetic fine powders, super paramagnetic ferrofluids (commercially available contrast medium Ferrumoxides injectable solution, Endoerm). Ferrofluids are water based colloidal suspensions of coated magnetic particles (coatings include anionic surfactant, starch or dextran). The coated particles can be suspended in water, aqueous formulations, and in liquid or solidified gels. The typical particle size is between 10 and 350 nm. In addition submicron magnetite particles with a cationic liposomal membrane (magnetic cationic liposomes) or magnetic particles with neutral liposome membranes (magnetoliposomes) can be used to selectively heat target tissue such as hair follicle without damaging the surrounding tissue(Cancer Sci. vol. 94, no. 9, pp. 834-39, 2003). An accurate temperature control can be accomplished by manipulating the magnetic field intensity (Cancer Sci. Vol. 94, No. 3, pp. 311-313, 2003). A method of devitalizing hair follicle tissue by using magnetically susceptible particles is described in U.S. Pat. No. 6,074,385.The skin also may be heated by applying nanoshells which in contrast to nanoparticles absorb light, for example metal nanoshells are intense absorbers of infrared and near-infrared light. The nanoshells absorb the infrared light and provides controlled, localized heating of the area of skin first treated with the nanoshells. Nanoshells and methods of heating nanoshells are disclosed, for example, in published U.S. Patent Applications 2002/0169235, 2002/0103517, and 2003/0118657, which are incorporated by reference herein. See also, Hirsch et al., PNAS, vol. 100, no. 23, pp. 13549-54. Specific examples of nanoshells that absorb infrared light include composite spherical nanoparticles consisting of a dielectric core covered by a thin metallic core which is typically gold (Technology in Cancer Research and Treatment Vol. 3, No. 1, 2004, pp. 33-40). The nanoparticles or nanoshells generally are applied to the skin in an appropriate vehicle. The vehicle may be, for example, a colloidal suspension, a liposomal formulation, an ointment, cream or a gel. The vehicle may also be an oil-in-water or water-in-oil emulsion formulated to a viscosity to match the density of the nanoparticle or nanoshell in order to keep these in a uniform suspension. The vehicle may include, for example, from 0.1% to 20% of the nanoparticles or nanoshells by weight.

The skin also can be heated using an exothermic heat dispensing material that is activated before contacting the skin. These materials are known and may be activated by kneading or by exposure to air (for example, by puncturing an inner bag including the material) to initiate a chemical reaction. The material may include, for example, a mixture of iron powder, water, cellulose, vermiculite activated carbon, and salt. This material can be enclosed in a laminated bag having an air permeable cloth layer and an impermeable film layer containing aeration holes; see U.S. Pat. No. 3,976,049, which is incorporated by reference. The material optionally may be included in a shaver or epilator.

Other mechanisms for heating the skin include using a microwave device; an ultrasound device; an ultrasound high frequency (radio frequency) device; a device including a flexible thermofoil; a light emitting diode device; a heater incorporated into a depilatory device; a battery operated heater device; and a device which produces warmed air or liquid that contacts the skin.

The area of skin may be, for example, on the face and may include the beard area of the face, i.e., the cheek, neck, upper lip, and chin; legs; arms; torso; armpits; or pubic area. The method can be used for reducing the growth of unwanted hair in women having hirsutism or other conditions as well as a method to enhance the benefits of shaving by decreasing the visibility of hair, five o'clock shadow, and promote the perception of a more long lasting shave or smoother skin. The method can be used in reducing the appearance and/or feel of the hair by making the hair thinner, softer, and less pigmented in addition to reducing its growth rate. The method may be used as an adjunct to other methods of hair removal including waxing, mechanical epilation, chemical depilation, electrolysis and laser-assisted hair removal.

In humans, the method can be used, for example, once or twice a day, to achieve a perceived reduction in hair growth. Perception of reduced hair growth could occur as early as 24 hours or 48 hours (for instance, between normal shaving intervals) following use or could take up to, for example, three months. Reduction in hair growth is demonstrated when, for example, the rate of hair growth is slowed, the need for removal is reduced, the subject perceives less hair on the treated site, or quantitatively, when the weight of hair removed (i.e., hair mass) is reduced.

The temperature of the skin obtained by heating by a particular mechanism generally can be determined as follows. A subject having a normal body temperature is placed in a room having a temperature of 25° C. A 0.009-inch-diameter thermocouple is placed in an area in the skin. The thermocouple output is connected to a National Instruments SCXI-1112 thermocouple signal conditioner. A National Instruments 6052E data acquisition board, having a maximum acquisition rate of 333 kilo-samples per second controlled the data acquisition and signal gain. The SCXI-1112 and NI 6052E DAQ combination could simultaneously detect up to eight thermocouple outputs at a rate of 42 kilo-samples per second. The sampling rate can be conducted, for example, at 1000 samples/sec.

A similar method is used to determine the temperature range in the hypodermal region. In this case, a thermocouple is inserted to a depth of about 5 mm in an ex-vivo human skin and treatment is applied at the skin surface.

The method also can be used in conjunction with application of compositions including one or more compounds that reduce the rate of hair growth. The hair growth inhibiting composition can be applied before, in conjunction with, or after the heat treatment. For example, a hair growth inhibiting composition can be applied from 24 hours to a week before the heat application.

The composition includes the compound(s) and a dermatologically acceptable vehicle. The composition may be a solid, semi-solid, or liquid. The composition may be, for example, a cosmetic and dermatologic product in the form of an, for example, ointment, lotion, foam, cream, gel, or solution. The composition may also be in the form of a shaving preparation or an aftershave. The vehicle itself can be inert or it can possess cosmetic, physiological and/or pharmaceutical benefits of its own.

Compounds that can reduce the ratio of hair growth, and compositions including the compounds, are described, for example, in Shander, U.S. Pat. No. 4,720,489; Breuer et al., U.S. Pat. No. 4,885,289; Ahluwalia, U.S. Pat. No. 5,095,007; Ahluwalia et al., U.S. Pat. No. 5,096,911; Shander et al., U.S. Pat. No. 5,132,293; Shander et al., U.S. Pat. No. 5,143,925; Shander et al., U.S. Pat. No. 5,328,686; Ahluwalia, U.S. Pat. No. 5,364,885; Shander et al., U.S. Pat. No. 5,411,991; Ahluwalia, U.S. Pat. No. 5,444,090; Ahluwalia et al., U.S. Pat. No. 5,455,234; Ahluwalia et al., U.S. Pat. No. 5,468,476; Shander et al., U.S. Pat. No. 5,474,763; Ahluwalia et al., U.S. Pat. No. 5,554,608; Boxall et al., U.S. Pat. No. 5,648,394; Henry et al., U.S. Pat. No. 5,652,273; Ahluwalia, U.S. Pat. No. 5,674,477; Shander et al., U.S. Pat. No. 5,728,736; Henry et al., U.S. Pat. No. 5,840,752; Henry et al., U.S. Pat. No. 5,908,867; Henry et al., U.S. Pat. No. 5,939,458; Styczynski et al., U.S. Pat. No. 5,958,946; Styczynski et al., U.S. Pat. No. 5,962,466; Styczynski et al., U.S. Pat. No. 6,020,006; Styczynski et al., U.S. Pat. No. 6,037,326; Styczynski et al., U.S. Pat. No. 6,060,471; Ahluwalia et al., U.S. Pat. No. 6,093,748; Henry et al., U.S. Pat. No. 6,121,269; Styczynski et al., U.S. Pat. No. 6,235,737; Ahluwalia et al., U.S. Pat. No. 6,239,170; Ahluwalia et al., U.S. Pat. No. 6,248,751; Styczynski et al., U.S. Pat. No. 6,299,865; and Ahluwalia et al., U.S. Pat. No. 6,414,017. These patents are incorporated by reference. Other compounds that can be used to reduce hair growth, and compositions including the compound(s) are described, for example in Styczynski et al., U.S. Ser. No. 09/893,252; Hwang et al., U.S. Ser. No. 10/145,283; U.S. Ser. No. 10/198,219; Styczynski et al., U.S. Ser. No. 10/347,987; Jardien et al., U.S. Ser. No. No. 10/397,132; Ahluwalia et al., U.S. Ser. No. 10/397,625; Hwang et al., U.S. Ser. No. 10/636,466; and Hwang et al., U.S. Ser. No. 10/721,118. All of these applications are owned by the owner of this application and are incorporated by reference.

The concentration of the compound in the composition may be varied over a wide range up to a saturated solution, preferably from 0.1% to 30% by weight or even more; the reduction of hair growth increases as the amount of compound applied increases per unit area of skin. The maximum amount effectively applied is limited only by the rate at which the compound penetrates the skin. The effective amounts may range, for example, from 10 to 3000 micrograms or more per square centimeter of skin.

The vehicle can be inert or can possess cosmetic, physiological and/or pharmaceutical benefits of its own. Vehicles can be formulated with liquid or solid emollients, solvents, thickeners, humectants and/or powders. Emollients include stearyl alcohol, mink oil, cetyl alcohol, oleyl alcohol, isopropyl laurate, polyethylene glycol, petroleum jelly, palmitic acid, oleic acid, and myristyl myristate. Solvents include ethyl alcohol, isopropanol, acetone, diethylene glycol, ethylene glycol, dimethyl sulfoxide, and dimethyl formamide.

The composition optionally can include components that enhance the penetration of the compound into the skin and/or to the site of action. Examples of penetration enhancers include urea, polyoxyethylene ethers (e.g., Brij-30 and Laureth-4), 3-hydroxy-3,7,1 1-trimethyl-1,6,10-dodecatriene, terpenes, cis-fatty acids (e.g., oleic acid, palmitoleic acid), acetone, laurocapram, dimethylsulfoxide, 2-pyrrolidone, oleyl alcohol, glyceryl-3-stearate, propan-2-ol, myristic acid isopropyl ester, cholesterol, and propylene glycol. A penetration enhancer can be added, for example, at concentrations of 0.1% to 20% or 0.5% to 5% by weight.

The composition also can be formulated to provide a reservoir within or on the surface of the skin to provide for a continual slow release of the compound. The composition also may be formulated to evaporate slowly from the skin, allowing the compound extra time to penetrate the skin.

Human Hair Follicle Growth Assay:

Human hair follicles in growth phase (anagen) were isolated from face-lift tissue (obtained from plastic surgeons) under dissecting scope using a scalpel and watchmakers forceps. The skin was sliced into thin strips exposing 2-3 rows of follicles that could readily be dissected. Follicles were placed into 0.5 ml William's E medium (Life Technologies, Gaithersburg, Md.) supplemented with 2 mM L-glutamine, 10 μg/ml insulin, 10 ng/ml hydrocortisone, 100 units of penicillin, 0.1 mg/ml streptomycin and 0.25 μg/ml amphotericin B. The follicles were incubated in 24-well plates (1 follicle/well) at 37° C. in an atmosphere of 5% CO₂ and 95% air. After the ‘heat’ treatment the individual follicles were placed into cell culture plates, one per well, and stored in the tissue culture incubator at 37° C. The follicles were photographed in the 24-well plates under the dissecting scope at a power of 10×. Typically, image recordings were made on day 0 (day follicles were placed in culture), and again on day 7. The length of hair follicle was assessed using an image analysis software system. The growth of hair fiber was calculated by the subtracting the follicle length on day 0 from that determined on day 7. A multi-scale stage micrometer (Titan Tool Supply Co., Inc.) was imaged at the same zoom factor as the follicles and served as the length calibration standard. The smallest division of the micrometer was 25 μm. The follicle length measurement was performed in IMAQ Vision Builder based on the micrometer calibration and stored in a Microsoft Excel file for further statistical analysis.

The catagen phase of the hair cycle is marked by the end of cell growth and pigmentation, loss of contact between the dermal papilla and matrix cell and shrinkage of the matrix area away from the dermal papilla by apoptosis. Catagen growth is measured based on the elongation of follicle bulb region, i.e., the distance between base of dermal papillae and hair shaft. Bulbs were defined to be in catagen when elongation was greater than or equal to the mean of high dose laser minus two standard errors of the mean.

The follicles (6 follicles at a time) were fully immersed in media that had been equilibrated to the specified (elevated) temperature with forceps. At the end of the specified time, the follicles were removed from the heated media and placed in either room temperature media or media that had been cooled to 0° C. Those follicles placed in room temperature media were left in the media until all the follicles had been treated and then placed in the 37° C. incubator. Those follicles that were cooled following treatment were placed in the 0° C. media for the specified time and then returned to room temperature media until all follicles had been treated.

### EXAMPLE 1

Follicles were placed in media at temperatures: room temperature (22-25° C.), 37° C., 45° C., 55° C., and 60° C. for one minute. Table 1 includes the measurements of follicle growth and elongation five days after treatment as shown. There was a slight decrease in growth when the follicles were incubated in the room temperature media, there was no effect with the 37° C. incubation (physiological hair follicle temperature in the hypodermal region. There was an almost 50% reduction at 45° C., a 70% reduction at 55° C. and complete inhibition of hair fiber growth at 60° C. There was no catagen development in these follicles. The catagen development was determined by the elongation of bulb region of the hair follicle from the base of the hair shaft to the base of the follicle.

TABLE 1

| | | Elongation of the |
|---|---|---|
| | Hair fiber growth | bulb region |
| Treatment | (mm ± SEM) | (mm ± SEM) |
| Control | 0.92 ± .07 | 0.02 ± .02 |
| Room temp | 0.71 ± .10 | 0.09 ± .05 |
| 37 | 0.94 ± .07 | 0.01 ± .02 |
| 45 | 0.49 ± .11 | 0.06 ± .04 |
| 55 | 0.27 ± .07 | 0.06 ± .05 |
| 60 | 0.09 ± 07 | −0.13 ± .04 |

### EXAMPLE 2

Follicles were heated for 10 seconds at 45° C., 50° C., 55° C., and 60° C. and checked for growth. The results are shown in Table 2. There was a 25% reduction in growth over the 5 days seen with heating at 45° C. Heating the follicle to 50° C. and 55° C. for 10 seconds was enough to decrease growth by 60%. Exposure of the follicles to 60° C. for 10 seconds was sufficient to completely inhibit hair growth. There was no evidence of catagen development in the follicles that had been heated for 10 seconds at these temperatures.

TABLE 2

| | | Elongation of the |
|---|---|---|
| | Hair fiber growth | bulb region |
| Treatment | (mm ± SEM) | (mm ± SEM) |
| Control | 0.80 ± .04 | 0.00 ± .02 |
| 45 | 0.58 ± .08 | 0.00 ± .03 |
| 50 | 0.34 ± .07 | 0.00 ± 0.03 |
| 55 | 0.34 ± .06 | 0.00 ± .02 |
| 60 | 0.00 ± .04 | 0.04 ± .02 |

### EXAMPLE 3

The follicles were exposed to heat for either 10 seconds or 30 seconds and then placed in media at 0° C. for 10 seconds. The placing of the follicles into the media at 0° C. had little or no effect on hair growth (see Table 3). There was no inhibition in hair growth until the follicles were heated at 60° C. It would seem that at lower temperatures that any changes that occur in the follicle at 40° C. and 50° C. can be reversed or prevented by cooling right after the heating process. However, the cooling of the follicle for 10 seconds is not enough to overcome the changes caused by heating the follicle for 10 seconds at 60° C.

TABLE 3

| | | | Elongation of |
|---|---|---|---|
| | Treatment for | Hair fiber Growth | the bulb region |
| Treatment | 10 sec. @) 0°-C. | (mm ± SEM) | (mm ± SEM) |
| Control | No | 0.76 ± .11 | 0.12 ± .07 |
| Cooling | Yes | 0.65 ± .11 | 0.05 ± .03 |
| Control | | | |
| 40° C. | Yes | 0.85 ± .12 | 0.10 ± 0.05 |
| 50 C. ° | Yes | 0.76 ± .08 | 0.01 ± .02 |
| 60° C. | Yes | 0.04 ± .03 | −0.01 ± .01 |

Finally, follicles were heated for 30 seconds and then cooled for 10 seconds at 0° C. The results are shown in Table 4.

TABLE 4

| | Heat for 30 sec., | | Elongation of the |
|---|---|---|---|
| | followed by | Hair fiber Growth | bulb region |
| Treatment | 0° C. for 10 sec. | (mm ± SEM) | (mm ± SEM) |
| Control | No | 1.04 ± .09 | 0.09 ± .03 |
| 45° C. | Yes | 0.71 ± .08 | −0.03 ± .02 |
| 50 C. ° | Yes | 0.49 ± .05 | −0.06 ± 0.02 |
| 55° C. | Yes | 0.25 ± .07 | −0.10 ± 0.02 |
| 60° C. | Yes | −0.06 ± .03 | −0.04 ± .01 |

Heating follicles for 30 seconds at 45° C. was sufficient to cause a 30% reduction in hair growth. The amount of hair growth inhibition increased with the increase in temperature the follicles were exposed to. Once the follicle is exposed to 60° C. for even short periods of time, cooling did not allow it to overcome the changes caused by heating.

To assess the effects of heating on the status of hair follicle cells the morphology and proliferation rate were analyzed. Twelve hair follicles per each group were analyzed. Histomorphology of the hair follicles (HF) exposed to +45° C. remained unchanged. However, hair follicles exposed to 45° C. heating contained a significantly (p<0.01) lower number of proliferating cells in the hair matrix (49 cells per HF), compared to control follicles (92 per HF), as determined by detection of PCNA. These observations suggest that heat (+45° C.) suppresses cell proliferation without visible alterations in hair follicle structure, leading to the retardation of hair shaft elongation. Normal hair growth rates appeared to resume within 72 hours, indicating that low heat provokes a transitory decrease in growth rate not accompanied by morphological or physiological effects associated with programmed cell death and hair cycle changes.

Compounds that reduce hair growth, such as alpha-difluoromethylomithine (DFMO), when used in combination with a laser, reduced the fluence threshold necessary for hair inhibition by a laser. Thus doses of DFMO not effective at inhibiting hair growth as well as a laser fluence not effective in mediating hair growth inhibition were demonstrated to act synergistically to promote hair growth inhibition when used in combination.

Other embodiments are within the claims.

What is claimed is:

1. A method of reducing hair growth in a mammal, comprising:
selecting an area of skin in a mammal from which hair grows according to a growth cycle and from which reduced hair growth of the hair is desired; and
heating said area of skin to a temperature sufficiently to retard the growth of the hair without altering the growth cycle of the hair.

2. The method of claim 1, wherein said area of skin is heated to a temperature of less than 55° C.

3. The method of claim 1, wherein said area of skin is heated to a temperature of between 40° and 50°.

4. The method of claim 2, wherein said area of skin is heated for at least a minute.

5. The method of claim 1, wherein said heating is performed at least twice in seven days.

6. The method of claim 1, wherein said heating is accomplished using a heater incorporated in a shaving device.

7. The method of claim 1, wherein said heating is accomplished using a laser.

8. The method of claim 1, wherein said heating is accomplished using a device that does not include a cooling element.

9. The method of claim 8, wherein the device is a laser.

10. The method of claim 8 wherein the device is an infrared source of light used in combination with nanoshells applied to skin surface prior to irradiation.

11. The method of claim 1, wherein the hair grows in response to androgen stimulation.

12. The method of claim 1, wherein the area of skin is on the face of a human.

13. The method of claim 1, wherein the area of skin is on the axilla or leg of a human.

14. The method of claim 1, further comprising, within 24 hours of said heating, applying to the area of skin a dermatologically acceptable composition including a compound that reduces the growth of hair.

15. The method of claim 14, wherein the compound is selected from the group consisting of alpha-difluoromethylornithine, triptolide, and caffeic acid phenyl ester.

16. The method of claim 14, wherein said compound is selected from the group consisting of inhibitors of angiogenesis and inhibitors of metalloproteinase.

17. A method of reducing hair growth in a mammal, comprising:
selecting an area of skin in a mammal from which hair grows and from which reduced hair growth of the hair is desired; and
heating said area of skin a temperature of between 40° C. and 55° C. to cause a reduction in the growth of the hair.

18. The method of claim 17, wherein said area of skin is heated to a temperature of wherein said area of skin is heated to a temperature of less than 52° C.

19. The method of claim 17, wherein said area of skin is heated to a temperature of wherein said area of skin is heated to a temperature of less than 50° C.

20. The method of claim 17, further comprising, within 24 hours of said heating, applying to the area of skin a dermatologically acceptable composition including a compound that reduces the growth of hair.

21. The method of claim 17 wherein said area of skin is heated to said temperature for at least a minute.

22 The method of claim 17 wherein said heating is performed at least twice in seven days.

23. The method of claim 17 wherein said heating is accomplished using a laser.

24. The method of claim 17 wherein said heating is accomplished using a heater incorporated into a shaving device.

25. The method of claim 17, wherein said heating is accomplished using a device that does not include a heating element.

26. The method of claim 17, wherein said area of skin is on the face of a human.
